(19) United States
(12) Patent Application Publication
(10) Pub. No.: US 2005/0192267 A1
(43) Pub. Date: Sep. 1, 2005

(54) Use of targeted oxidative therapeutic formulation in treatment of viral diseases

(76) Inventor: Robert F. Hofmann, Austin, TX (US)

Correspondence Address: JACKSON WALKER LLP, 2435 NORTH CENTRAL EXPRESSWAY, SUITE 600, RICHARDSON, TX, 75080, US

(21) Appl. No.: 11/059,947

(22) Filed: Feb. 17, 2005

Related U.S. Application Data
(60) Provisional application No. 60/546,350, filed on Feb. 20, 2004.

(51) Int. Cl. A61K 31/555; A61K 31/525; A61K 31/045; A61K 31/12; A61K 31/353

(52) U.S. Cl. 514/185; 514/724; 514/251; 514/454; 514/680; 514/410

(57) ABSTRACT

A pharmaceutical formulation and its use. The pharmaceutical formulation contains peroxidic species or reaction products resulting from oxidation of an alkene, such as geraniol, by an oxygen-containing oxidizing agent, such as ozone; a penetrating solvent, such as dimethylsulfoxide (“DMSO”); a dye containing a chelated metal, such as hematoporphyrin; and an aromatic redox compound, such as benzoquinone. The pharmaceutical formulation is used to effectively treat patients affected with viruses such as Hepatitis C and HIV-1.

### BACKGROUND

This application claims priority to U.S. Provisional Patent Application, Ser. No. 60/546,350, entitled “USE OF TARGETED OXIDATIVE THERAPEUTIC FORMULATION IN TREATMENT OF VIRAL DISEASES” filed on Feb. 20,2004, having Robert F. Hofmann, listed as the inventor, the entire content of which is hereby incorporated by reference.

The present invention relates to a composition containing peroxidic species or oxidation products, its method of preparation, and its use. More specifically, the invention relates to a pharmaceutical composition or formulation which contains: peroxidic species or reaction products resulting from oxidation of an olefinic compound, in a liquid form or in a solution, by an oxygen-containing oxidizing agent; a penetrating solvent; a dye containing a chelated metal; and an aromatic redox compound. The invention also relates to the preparation of the pharmaceutical formulation and its use in treating viral diseases.

Ozone is a triatomic gas molecule and an allotropic form of oxygen. It may be obtained by means of an electrical discharge or intense ultraviolet light through pure oxygen. The popular misconception that ozone is a serious pollutant, the “free radical” theory of disease, and the antioxidant supplement market have comprehensibly prejudiced medical orthodoxy against its use as a treatment. Ozone therapy, however, is a misnomer. Ozone is an extremely reactive and unstable gas with mechanisms of action directly related to the by-products that it generates through selective interaction with organic compounds present in the plasma and in the cellular membranes. The selective reaction of ozone with unsaturated olefins occurs at the carbon-carbon double bond, generating ozonides. Ozone is toxic by itself, and its reaction products, ozonides, are unstable and are not therapeutic by themselves.

Hydrogen peroxide (H₂O₂), discovered in 1818, is present in nature in trace amounts. Hydrogen peroxide is unstable and decomposes violently (or foams) when in direct contact with organic membranes and particulate matter. Light, agitation, heating, and iron all accelerate the rate of hydrogen peroxide decomposition in solution. Hydrogen peroxide by direct contact ex vivo kills microbes that have low levels of peroxide-destroying enzymes, such as the catalases. However, there is no bactericidal effect when hydrogen peroxide is infused into the blood of rabbits infected with peroxide-sensitive E. coli. Moreover, increasing the concentration of peroxide ex-vivo in rabbit or human blood containing E. coli produces no evidence of direct bactericidal activity. The lack of effect of high concentrations of hydrogen peroxide is directly related to the presence of the peroxide-destroying enzyme catalase in the host animal's blood. To have any effect, high concentrations of hydrogen peroxide have to be in contact with the bacteria for significant periods of time. Large amounts of hydrogen peroxide-destroying enzymes, such as catalase, normally present in the blood make it impossible for peroxide to exist in blood for more than a few seconds. Thus, hydrogen peroxide introduced into the blood stream by injection or infusion does not directly act as an extracellular germicide in blood or extracellular fluids.

However, hydrogen peroxide does participate in the bactericidal processes of activated macrophage cells. Activated macrophage cells are drawn to the site of infection, attach to the infectious organism, and ingest it. The killing of the organisms takes place inside the macrophage cell by hydrogen peroxide. Hydrogen peroxide oxidizes cellular chloride to the chlorine dioxide free radical, which destabilizes microbial membranes and, if persistent, induces apoptosis or cellular suicide. The critical therapeutic criteria for intracellular peroxidation are the selective delivery, absorption and activation of peroxidic carrier molecules into only diseased macrophages, which are believed to be incapable of upgraded catalase and glutathione reductase activity. Infused hydrogen peroxide is a generalized poison whereas targeted intracellular peroxidation is a selective therapeutic tool.

Macrophage cells play critical roles in immunity, bone calcification, vision, neural insulation (myelinization), detoxification, pump strength, and clearance of toxins from the body, depending upon their site of localization. The energy requirements of macrophages are met by intracellular structures called mitochondria. Mitochondria are often structurally associated with the microfilament internal cytoarchitecture. The folded internal layer of the mitochondria creates the high-energy molecule ATP, while the outer layer contains cytochromes and electron recycling molecules that generate peroxides. The outer layers of mitochondria are susceptible to toxic blockade or damage by endotoxins, mycotoxins, drugs, heavy metals, and pesticides. When the peroxidation function of mitochondria is blocked, the filament architecture of the cell tends to cross-link, generating incorrect signals, incompetence, inappropriate replication, or premature cell death.

The mitochondrial cytochrome oxidase enzyme activity is markedly reduced in many malignant tumors and virus-infected macrophages. (Allen, et al., 1977). In particular, studies of simian viral-transformed and non-transformed cells have shown that the activity of the mitochondrial cytochrome oxidase enzyme in transformed cells was only 50% of the activity in non-transformed cells. (White, et al., 1975).

Two viruses having a great impact on public health today are Hepatitis C Virus (“HCV”) and Human Immunodeficiency Virus (“HIV-1”). Both HCV and HIV-1 are RNA viruses. The structure of RNA viruses is basically the same as that of other viruses: a core of genetic material, usually contained within a protective capsid of protein, and in many cases, a lipid envelope as well. The life cycle of the RNA viruses is also similar: attachment to the host cell, penetration, reproduction of genetic material, creation of the protective capsid, and emergence from the cell. The major differences arise from the fact that the RNA viruses genetic information is stored, as their name suggests, in RNA, not DNA. RNA viruses are simple, requiring only small amounts of genetic material to encode the information necessary for their survival and requiring no additional enzymes to be packaged into their cores.

Hepatitis C virus (“HCV”) is one of the viruses (A, B, C, D, and E), which together account for the vast majority of cases of viral hepatitis. It is an enveloped RNA virus in the flaviviridae family, which has a narrow host range. Humans and chimpanzees are the only known species susceptible to infection, with both species developing similar disease. An important feature of the virus is the relative mutability of its genome, which in turn is related to the high propensity (80%) of inducing chronic infection. HCV is clustered into several distinct genotypes.

HCV is spread primarily by direct contact with human blood. Transmission has occurred through blood transfusions that were not screened for HCV infection, through the reuse of inadequately sterilized needles, syringes or other medical equipment, and through needle sharing among drug-users. Sexual and perinatal transmission may also occur, although less frequently. Other modes of transmission occur as a result of social, cultural, and behavioral practices using percutaneous procedures (e.g. ear and body piercing, circumcision, tattooing), if inadequately sterilized equipment is used. HCV is not spread by sneezing, hugging, coughing, food or water, sharing eating utensils, or casual contact.

The early symptoms of Hepatitis C are difficult to recognize because they are progressive in nature and often very mild. For more than six months following initial infection, the disease is virtually undetectable. The most common symptom, commencing sometimes a decade after initial infection, is fatigue. Other symptoms include mild fever, muscle and joint aches, nausea, vomiting, loss of appetite, vague abdominal pain, and sometimes, diarrhea. Many cases go undiagnosed because the symptoms are suggestive of a flu-like illness, which just comes and goes, or these symptoms are so mild that the patient is unaware of anything unusual. A minority of patients notices dark urine and light colored stools, followed by jaundice in which the skin and whites of the eyes appear yellow. Itching of the skin may be present. Some affected individuals may lose 5 to 10 pounds.

Individuals infected with HCV are often identified because they are found to have elevated liver enzymes on a routine blood test or because a Hepatitis C antibody is found to be positive at the time of blood donation. In general, elevated liver enzymes and a positive antibody test for HCV (anti-HCV) means that an individual has chronic Hepatitis C. A very small percentage of patients may recover from acute Hepatitis C, but their anti-HCV test will remain positive thereafter. Low-level infection, in which the infected individual is virtually asymptomatic but still highly contagious, may continue for years, even decades, before progressing significantly. However, more than 80% of infected individuals eventually progress to the chronic stage of the disease, which eventually results in cirrhosis (scarring of the liver tissue), and end-stage liver disease. This final cirrhotic stage takes, on average, about 20 years to develop.

At this pre-terminal stage, the symptoms are commensurate with liver failure, including jaundice and abdominal swelling (due to fluid retention called ascites), depending on the severity of the liver disease and whether or not cirrhosis has developed. Some patients with cirrhosis do well over time, while others die in ten and sometimes five years. Disorders of the thyroid, intestine, eyes, joints, blood, spleen, kidneys and skin may occur in about 20% of patients. Primary liver cancer can also develop from Hepatitis C, a late risk factor, which may be present 30 years or so after infection.

The therapy of chronic Hepatitis C has evolved steadily since alpha interferon was first approved for use in this disease more than ten years ago. Antiviral drugs such as interferon taken alone, or in combination with ribavirin, can be used for the treatment of persons with chronic Hepatitis C. Treatment with interferon alone is effective in about 10% to 20% of patients. Interferon combined with ribavirin is effective in about 30% to 50% of patients. Ribavirin does not appear to be effective when used alone. Existing therapies are most effective in patients with Genotypes 2 and 3, which represent about 25 percent of the patients in the U.S. Genotypes 1a and 1b, which affect about 75% of patients, are the most difficult to treat.

The optimal approved regimen is typically a 24- or 48-week course of the combination of pegylated alpha interferon and ribavirin (Manns, et al., 2001). Side effects of interferon and ribavirin treatments include fatigue, depression, headaches, nausea and vomiting, skin irritation, irritability, and sinusitis.

What is needed, therefore, is a method for treating viruses such as Hepatitis C which is effective and does not produce pronounced side effects.

HIV-1 is a fairly complex virus, thought to contain 2 identical copes of a positive sense (i.e. MRNA) single-stranded RNA strand about 9,500 nucleotides long. These may be linked to each other to form a genomic RNA dimer.

HIV-1 causes disease by infecting the CD4+T cells. These are a subset of leukocytes (white blood cells) that normally coordinate the immune response to infection. By using CD4+T cells to replicate itself, HIV-1 spreads throughout the body and at the same time depletes the cells that the body needs to fight the virus. Once a HIV positive individual's CD4+T cell count has decreased to a certain threshold, they are prone to a range of diseases that the body can normally control. HIV-infected individuals who are at serious risk of these opportunistic infections are said to have Acquired Immunodeficiency Syndrome (“AIDS”).

More than 830,000 cases of AIDS have been reported in the United States since 1981. As many as 950,000 Americans may be infected with HIV-1, one-quarter of whom are unaware of their infection. The epidemic is growing most rapidly among minority populations and is a leading killer of African-American males ages 25 to 44. According to the U.S. Centers for Disease Control and Prevention (“CDC”), AIDS affects nearly seven times more African Americans and three times more Hispanics than whites.

Many people do not have any symptoms when they first become infected with HIV-1. Some people, however, have a flu-like illness within a month or two after exposure to the virus. This illness may include fever, headache, tiredness, and enlarged lymph nodes. These symptoms usually disappear within a week to a month and are often mistaken for those of another viral infection. During this period, people are very infectious. More persistent or severe symptoms may not appear for 10 years or more after HIV-1 first enters the body in adults, or within two years in children born with HIV-1 infection. This period of “asymptomatic” infection is highly individual. Some people may begin to have symptoms within a few months, while others may be symptom-free for more than 10 years.

As the immune system worsens, a variety of complications start to take over. For many people, the first signs of infection are large lymph nodes or “swollen glands” that may be enlarged for more than three months. Other symptoms often experienced months to years before the onset of AIDS include lack of energy, weight loss, frequent fevers and sweats, persistent or frequent yeast infections (oral or vaginal), persistent skin rashes or flaky skin, pelvic inflammatory disease in women, and short-term memory loss. Some people develop frequent and severe herpes infections that cause mouth, genital, or anal sores, or a painful nerve disease called shingles.

HIV-positive patients today are given a complex regime of drugs that attack HIV-1 at various stages in its life cycle. These are known as anti-retroviral drugs and include protease inhibitors, reverse transcriptase inhibitors, and entry inhibitors. Many problems are involved in establishing a course of treatment for HIV-1. Each effective drug comes with side effects, often serious and sometimes life-threatening in themselves. Common side effects include extreme nausea and diarrhea, liver damage and failure, and jaundice. Any treatment requires regular blood tests to determine continued efficacy (in terms of T-cell count and viral load) and liver function.

What is needed, therefore, is a method for treating viruses such as HIV-1 which is effective and does not produce pronounced side effects.

U.S. Pat. No. 4,451,480 to De Villez teaches a composition and method for treating acne. The method includes topically treating the affected area with an ozonized material derived from ozonizing various fixed oil and unsaturated esters, alcohols, ethers and fatty acids.

U.S. Pat. No. 4,591,602 to De Villez shows an ozonide of Jojoba used to control microbial infections.

U.S. Pat. No. 4,983,637 to Herman discloses a method to parenterally treat local and systemic viral infections by administering ozonides of terpenes in a pharmaceutically acceptable carrier.

U.S. Pat. No. 5,086,076 to Herman shows an antiviral composition containing a carrier and an ozonide of a terpene. The composition is suitable for systemic administration or local application.

U.S. Pat. No. 5,126,376 to Herman describes a method to topically treat a viral infection in a mammal using an ozonide of a terpene in a carrier.

U.S. Pat. No. 5,190,977 to Herman teaches an antiviral composition containing a non-aqueous carrier and an ozonide of a terpene suitable for systemic injection.

U.S. Pat. No. 5,190,979 to Herman describes a method to parenterally treat a medical condition in a mammal using an ozonide of a terpene in a carrier.

U.S. Pat. No. 5,260,342 to Herman teaches a method to parenterally treat viral infections in a mammal using an ozonide of a terpene in a carrier.

U.S. Pat. No. 5,270,344 to Herman shows a method to treat a systemic disorder in a mammal by applying to the intestine of the mammal a trioxolane or a diperoxide derivative of an unsaturated hydrocarbon which derivative is prepared by ozonizing the unsaturated hydrocarbon dissolved in a non-polar solvent.

U.S. Pat. No. 5,364,879 to Herman describes a composition for the treatment of a medical condition in a mammal, the composition contains a diperoxide or trioxolane derivative of a non-terpene unsaturated hydrocarbon which derivative is prepared by ozonizing below 35° C. the unsaturated hydrocarbon in a carrier.

Despite the reports on the use of terpene ozonides for different medical indications, terpene ozonides display multiple deficiencies. For example, ozonides of monoterpene, such as myrcene and limonene, flamed out in the laboratory. Consequently, they are extremely dangerous to formulate or store.

Furthermore, ozonides of geraniol, a linear monoterpene alcohol, in water or in dimethylsulfoxide (“DMSO”) did not show any clinical efficacy in three cases of viral Varicella Zoster (shingles) and two cases of Herpes Simplex dermatitis.

Thus, there is a need for a safe and effective pharmaceutical formulation or composition utilizing reaction products from the oxidation of an alkene compound. What is also needed is a method for stimulating mitochondrial defenses against free radical formation and effectively treating individuals infected with viruses such as Hepatitis C and HIV-1.

### SUMMARY

This invention is directed to pharmaceutical formulations comprising peroxidic species or reaction products resulting from oxidation of an unsaturated organic compound, in a liquid form or in a solution, by an oxygen-containing oxidizing agent; a penetrating solvent; a chelated dye; and an aromatic redox compound. In one embodiment of the pharmaceutical formulation, the essential components include the peroxidic products formed by ozonolysis of an unsaturated alcohol, a stabilizing solvent, metalloporphyrin, and quinone. This invention is also directed to use of the pharmaceutical formulation to treat viruses.

The peroxidic species or reaction products are preferably formed through the reaction of an alkene and ozone. It is generally accepted that the reaction between an alkene and ozone proceeds by the Criegee mechanism. According to this mechanism, shown in Scheme 1 below, the initial step of the reaction is a 1,3-dipolar cycloaddition of ozone to the alkene to give a primary ozonide (a 1,2,3-trioxalane). The primary ozonide is unstable, and undergoes a 1,3-cycloreversion to a carbonyl compound and a carbonyl oxide. In the absence of other reagents or a nucleophilic solvent, this new 1,3-dipole enters into a second 1,3-dipolar cycloaddition to give the “normal” ozonide, a 1,2,4-trioxalane.

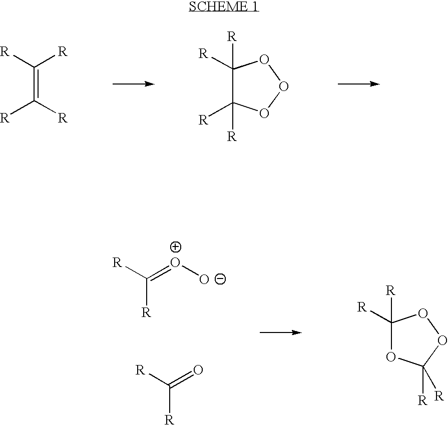

In a side reaction, the carbonyl oxide can enter into a dimerization to give a peroxidic dimer, the 1,2,4,5-tetraoxane, shown in Scheme 2 below.

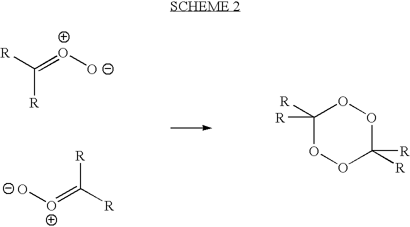

The carbonyl oxide is a strongly electrophilic species, and in the presence of nucleophilic species (e.g. alcohols or water), it undergoes facile nucleophilic addition to give a 1-alkoxyhydroperoxide, shown in Scheme 3 below. Under certain conditions, the 1-alkoxyhydroperoxide can undergo further reaction to give carboxylic acid derivatives.

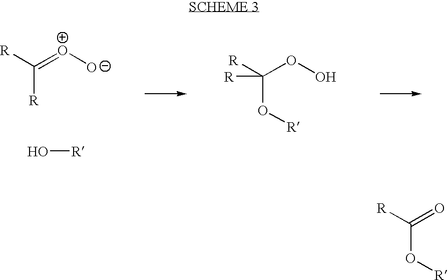

Again, not wanting to be bound by theory, it is believed that during the ozonolysis of the alcohol-containing alkene in the present invention, it is reasonable to expect that three major types of peroxidic products will be present: the normal ozonide, the carbonyl tetraoxane dimer, and the 1-alkoxyhydroperoxide. In the presence of water, some of these peroxidic products may also lead to the presence of organic peracids in the crude product mixture.

The present invention also involves the use of a penetrating solvent such as dimethylsulfoxide (“DMSO”) to “stabilize” the initial products of the ozonolysis. Similarly, not wanting to be bound by any theory, it is believed that the stabilization is most likely a simple solvation phenomenon. However, DMSO is known to be a nucleophile in its own right. Its participation is also possible as a nucleophilic partner in stabilizing reactive species (for example, as dimethylsulfoxonium salts). The stabilized peroxidic molecule and the penetrating solvent of the current pharmaceutical formulation are made from components generally regarded as safe (“GRAS”).

Another component of the pharmaceutical formulation is a chelated dye, such as a porphyrin. The propensity of metalloporphyrins to sensitize oxygen under photochemical excitation is well documented, as is the propensity of ferroporphyrins and copper porphyrins to bind oxygen-containing systems.

A further component of the pharmaceutical formulation is an aromatic redox compound, such as a quinone.

Although not wanting to be bound by any theory, it is postulated that the preferred pharmaceutical formulation is a combination of biochemical agents that induce recycling autocatalytic oxidation in infected or dysplastic macrophages. The pharmaceutical formulation stimulates targeted apoptosis (cell suicide) through unopposed peroxidation. Thus, the pharmaceutical formulation creates therapeutic effects in a number of seemingly disparate mitochondria-based macrophagic diseases. In particular, the pharmaceutical formulation has been shown to be effective in individuals infected with Hepatitis C virus (“HCV”) by reducing viral RNA levels, restoring normal liver enzyme levels, and improving overall symptoms. The pharmaceutical formulation is also effective at reducing viral replication rates in vitro. These results indicate its effectiveness at treating viruses.

### BRIEF DESCRIPTION OF FIGURES

FIG. 1 shows the infectious units present in supernatant taken from cultured astrocyte cells infected with a murine retrovirus. Results from cells treated with two concentrations of two samples of the pharmaceutical formulation are shown, along with results for untreated, infected control cells.

FIG. 2 shows the viable cell count of cultured astrocyte cells treated with two concentrations of two samples of the pharmaceutical formulation, along with the viable cell count for untreated, uninfected control cells.

FIG. 3 shows the viable cell count of cultured astrocyte cells infected with a murine retrovirus, after treatment with two concentrations of two samples of the pharmaceutical formulation. Results for untreated, infected control cells are also shown.

### DETAILED DESCRIPTION OF PREFERRED EMBODIMENTS

The current invention pertains to pharmaceutical formulations comprising peroxidic species or reaction products resulting from oxidation of an unsaturated organic compound, in a liquid form or in a solution, by an oxygen-containing oxidizing agent; a penetrating solvent; a chelated dye; and an aromatic redox compound. The pharmaceutical formulations may be used for antiviral purposes, such as treatment of Hepatitis C virus (“HCV”) and HIV-1. In one embodiment of the present invention, the essential components of the pharmaceutical formulation include the peroxidic products formed by ozonolysis of an unsaturated alcohol, a stabilizing solvent, metalloporphyrin, and quinone.

The unsaturated organic compound, which may also be an unsaturated olefinic hydrocarbon, of the pharmaceutical formulation can be an alkene without a hydroxyl group, or a hydroxyl-containing alkene. Preferably, the alkene has less than about 35 carbons. The alkene without a hydroxyl group may be an open-chain unsaturated hydrocarbon, a monocyclic unsaturated hydrocarbon, or a bicyclic unsaturated hydrocarbon. The hydroxyl-containing alkene can be an open-chain unsaturated alcohol, a monocyclic unsaturated alcohol, or a bicyclic unsaturated alcohol. The alkene may also be contained in a fixed oil, an ester, a fatty acid, or an ether.

Usable unsaturated olefinic hydrocarbons may be unsubstituted, substituted, cyclic or complexed alkenes, hydrazines, isoprenoids, steroids, quinolines, carotenoids, tocopherols, prenylated proteins, or unsaturated fats. The preferred unsaturated hydrocarbons for this invention are alkenes and isoprenoids.

Isoprenoids are found primarily in plants as constituents of essential oils. While many isoprenoids are hydrocarbons, oxygen-containing isoprenoids also occur such as alcohols, aldehydes, and ketones. In a formal sense, the building block of isoprenoid hydrocarbons may be envisaged as the hydrocarbon isoprene, CH₂═C(CH₃)—CH═CH₂, although it is known that isoprene itself is an end-product of isoprenoid biosynthesis and not an intermediate. Isoprenoid hydrocarbons are categorized by the number of isoprene (C₅H₈) units they contain. Thus, monoterpenes have 2, sesquiterpenes have 3, diterpenes have 4, sesterterpenes have 5, triterpenes have 6, and tetraterpenes have 8 isoprene units, respectively. Tetraterpenes are much more commonly known as carotenoids.

Limonene and pinene are examples of a monoterpene. Famesol and nerolidol are examples of a sesquiterpene alcohol. Vitamin A₁ and phytol are examples of a diterpene alcohol while squalene is an example of a triterpene. Provitamin A₁, known as carotene, is an example of a tetraterpene. Geraniol, a monoterpene alcohol, is liquid in both its oxygen bound and normal states and is safe to living cells.

Preferred unsaturated hydrocarbons for the pharmaceutical formulation include alkene isoprenoids, such as myricene, citrillene, citral, pinene, or limonene. Preferred unsaturated hydrocarbons also include linear isoprenoid alcohols with two to four repeating isoprene groups in a linear chain, such as α-terpineol, citronellol, nerol, phytol, menthol, geraniol, geranylgeraniol, linalool, or famesol.

The unsaturated organic compound maybe linear, branched, cyclic, spiral, or complexed with other molecules in its configuration. The unsaturated organic compound may naturally exist in a gaseous liquid or solid state prior to binding with the oxidizing agent.

An open-chain unsaturated hydrocarbon can be: CₙH₂ₙ, one double bond, n=2-20; CₙH₂ₙ₋₂, two double bonds, n=4-20; CₙH₂ₙ₋₄, three double bonds, n=6-20; CₙH₂ₙ₋₆, four double bonds, n=8-20; C₂₅H₄₀, sesterterpene hydrocarbon; or C₃₀H₄₈, triterpene hydrocarbon.

A monocyclic unsaturated hydrocarbon can be: CₙH₂ₙ₋₂, one double bond and one ring, n=3-20; CₙH₂ₙ₋₄, two double bonds and one ring, n=5-20; CₙH₂ₙ₋₆, three double bonds and one ring, n=7-20; C₂₅H₄₀, sesterterpene hydrocarbon; or C₃₀H₄₈, triterpene hydrocarbon.

A bicyclic unsaturated hydrocarbon can be: CₙH₂ₙ₋₄, one double bond and two rings, n=4-20; CₙH₂ₙ₋₆, two double bonds and two rings, n=6-20; C₂₅H₄₀, sesterterpene hydrocarbon; or C₃₀H₄₈, triterpene hydrocarbons.

An open-chain unsaturated alcohol can be: CₙH₂ₙOₘ, one double bond, n=3-20, m=1-4; CₙH₂ₙ₋₂Oₘ, two double bonds, n=5-20, m=1-4; CₙH₂ₙ₋₄Oₘ, three double bonds, n=7-20, m=1-4; CₙH₂ₙ₋₆Oₘ, four double bonds, n=9-20, m=1-4; C₂₅H₄₀Oₘ, m=1-4, sesterterpene alcohols; or C₃₀H₄₈Oₘ, m=1-4, triterpene alcohols.

A monocyclic unsaturated alcohol can be: CₙH₂ₙ₋₂Oₘ, one double bond and one ring, n=3-20, m=1-4; CₙH₂ₙ₋₄Oₘ, two double bonds and one ring, n=5-20, m=1-4; CₙH₂ₙ₋₆Oₘ, three double bonds and one ring, n=7-20, m=1-4; C₂₅H₄₀Oₘ, m=1-4, sesterterpene alcohols; or C₃₀H₄₈Oₘ, m=1-4, triterpene alcohols.

A bicyclic unsaturate alcohol can be: CₙH₂ₙ₋₄Oₘ, one double bond and two rings, n=5-20, m=1-4; CₙH₂ₙ₋₆Oₘ, two double bonds and two rings, n=7-20, m=1-4; C₂₅H₄₀Oₘ, m=1-4, sesterterpene alcohols; or C₃₀H₄₈Oₘ, m=1-4, triterpene alcohols.

Based on the total weight of the pharmaceutical formulation, the alkene can vary from about 0.001% to about 30%, preferably from about 0.1% to about 5.0%, and more preferably from about 0.5% to about 3.0%.

The oxygen-containing oxidizing agent of the pharmaceutical formulation, which oxidizes the unsaturated hydrocarbon, may be singlet oxygen, oxygen in its triplet state, superoxide anion, ozone, periodate, hydroxyl radical, hydrogen peroxide, alkyl peroxide, carbamyl peroxide, benzoyl peroxide, or oxygen bound to a transition element, such as molybdenum (e.g. MoO₅).

The preferred method to bind “activated oxygen” to intact an isoprenoid alcohol, such as geraniol, is by ozonation at temperatures between 0-20° C. in the dark in the absence of water or polar solvent. The geraniol “ozonides” are then dissolved and stabilized in 100% DMSO in the dark to prevent premature breakdown of the products. Although not wanting to be bound by any theory, it is believed that the catalytic breakdown of the tetraoxane peroxidic dimer byproduct of geraniol ozonation, which is not an ozonide, occurs inside of cells in the presence of superoxide anion. The final reactive therapeutic agents released are hydrogen peroxide and acetic acid.

The pharmaceutical formulation also utilizes a penetrating solvent. The penetrating solvent, which stablizes the oxygen-bound unsaturated hydrocarbon, may be an emollient, a liquid, a micelle membrane, or a vapor. Usable penetrating solvents include aqueous solution, fats, sterols, lecithins, phosphatides, ethanol, propylene glycol, methylsulfonylmethane, polyvinylpyrrolidone, pH-buffered saline, and dimethylsulfoxide (“DMSO”). The preferred penetrating solvents include DMSO, polyvinylpyrrolidone, and pH-buffered saline. The most preferred penetrating solvent is DMSO.

Based on the total weight of the pharmaceutical formulation, the penetrating solvent can vary from about 50% to about 99%, preferably from about 90% to about 98%, and more preferably from about 95% to about 98%.

The “stabilized” peroxidic molecule and its penetrating solvent have been made from components currently used in production regulated by the Food and Drug Administration (“FDA”). These ingredients are the subject of Drug Master Files, Drug Monographs, are found in the USP/NF, or are Generally Recognized As Safe (“GRAS”).

Another component of the pharmaceutical formulation is a chelated dye. The dye preferably contains a chelated divalent or trivalent metal, such as iron, copper, manganese tin, magnesium, or strontium. The preferred chelated metal is iron. The propensity of chelated dyes such as metalloporphyrins to sensitize oxygen under photochemical excitation is well documented, as is the propensity of ferroporphyrins and copper porphyrins to bind oxygen-containing systems. Usable dyes include natural or synthetic dyes. Examples of these dyes include porphyrins, rose bengal, chlorophyllins, hemins, porphins, corrins, texaphrins, methylene blue, hematoxylin, eosin, erythrosin, flavinoids, lactoflavin, anthracene dyes, hypericin, methylcholanthrene, neutral red, and fluorescein. Preferred dyes can be any natural or synthetic porphyrin, hematoporphyrin, chlorophyllin, rose bengal, their respective congeners, or a mixture thereof. The most preferred dyes are mixtures of hematoporphyrin and rose bengal, and mixtures of hematoporphyrin and chlorophyllin. The dye may be responsive to photon, laser, ionizing radiation, phonon, electrical cardiac electroporation, magnetic pulse, or continuous flow excitation.

Based on the total weight of the pharmaceutical formulation or composition, the dye can vary from about 0. 1% to about 30%, preferably from about 0.5% to about 5%, and more preferably from about 0.8% to about 1.5%.

A further component of the pharmaceutical formulation is an aromatic redox compound, such as a quinone. The aromatic redox compound may be any substituted or unsubstituted benzoquinone, naphthoquinone, or anthroquinone. Preferred aromatic redox compounds include benzoquinone, methyl-benzoquinone, naphthoquinone, and methyl-naphthoquinone. The most preferred aromatic redox compound is methyl-naphthoquinone.

Based on the total weight of the pharmaceutical formulation, the aromatic redox compound can vary from about 0.01% to about 20.0%, preferably from about 0.1% to about 10%, and more preferably from about 0.1% to about 0.5%.

The pharmaceutical formulation is also preferably activated by an energy source or an electron donor. Useful electron donors include an electrical current, ascorbate or ascorbic acid, NADH or NADPH, and germanium sesquioxide. Preferred electron donors include ascorbate and NADH. The most preferred electron donor is ascorbic acid in any salt form.

Based on the total weight of the pharmaceutical formulation, the electron donor can vary from about 0.01% to about 20%, preferably from about 1% to about 10%, and more preferably from about 1% to about 5%.

In order to obtain a biological effect in vivo, the pharmaceutical formulation is preferably infused as an ozonolysis-generated peroxidic product of an unsaturated hydrocarbon, rather than an ozonide, in conjunction with a superoxide generating chelated dye and an aromatic quinone. The unsaturated hydrocarbon product, or peroxidic dimer molecule, should be stabilized in a non-aqueous stabilizing solvent and should be capable of penetrating lipid membranes.

Researchers of energetically activated dye therapy have long known that the superoxide generating dye and the aromatic redox compound preferentially absorb into infected and dysplastic cells, which are typically also catalase deficient. Without wanting to be bound by theory, the catalase-induced destruction of peroxide should be overwhelmed in the target cells either naturally or by the pharmaceutical formulation. The peroxidic dimer should also be activated by the superoxide generating dye, initiating electron donation to the dimer and causing the release of hydrogen peroxide and acetic acid intracellularly. The electronic activation of the dye does not always require light, but rather may occur through small electrical pulses provided by, for example, a heart pulse. The peroxidation reaction within the infected macrophage then tends to destroy the prenylated protein linkage of microtubules within the cell, to destroy the infecting toxin, or to induce apoptosis of the macrophage host cell.

The pharmaceutical formulation is a combination of stable ingredients. These ingredients may preferably be stored as dry solid ingredients and liquid ingredients in separate containers, which are then mixed at the site of use. The dry solid ingredients preferably comprise the chelated dye and the aromatic redox compound. The liquid ingredients preferably comprise the peroxidic species or reaction products resulting from oxidation of the unsaturated hydrocarbon by the oxygen-containing active agent, along with the penetrating solvent. Administration is preferably intravenously. The reconstituted product preferably may be administered intravenously as a concentrate diluted in saline. Endodontic and intrathecal deliveries are also possible routes for administration. Intramuscular injection is not preferred, as it has a tendency to produce local irritation.

Administration of the pharmaceutical formulation in vivo is effective in treating viruses and symptoms of viruses in affected patients. In particular, the pharmaceutical formulation reduces viral RNA levels, restores normal liver enzyme levels, and improves overall symptoms of patients infected with Hepatitis C virus (“HCV”). The pharmaceutical formulation also reduces viral replication rates, including that of the murine retrovirus MoMuLV, in vitro. The pharmaceutical formulation also inhibits the infection and replication of HIV-1 in human CD4+ cells. The pharmaceutical formulation is also non-toxic to cells.

### EXAMPLE 1

### Ozonolysis of an Unsaturated Hydrocarbon

Ozonolysis of an alkene may be carried out either in a solvent or neat. In either case, the cooling of the reaction mixture is critical in avoiding explosive decomposition of the peroxidic products of the reaction.

The following general procedure is typical for the ozonolysis of a liquid alkene.

A 1-liter flask fitted with a magnetic stirrer is charged with the alkene (2 moles), and the apparatus is weighed. The flask is surrounded by a cooling bath (ice-water or ice-salt). Once the contents are cooled below 5° C., stirring is begun and a stream of ozone in dry oxygen (typically 3% ozone) is passed through the mixture. It is advantageous to disperse the ozonated oxygen through a glass frit, but this is not necessary for a stirred solution. Periodically, the gas stream is stopped, and the reaction flask is weighed or the reaction mixture is sampled. The gas stream is then re-started.

Once the mass of the reaction flask shows sufficient weight gain, or once the proton magnetic resonance (“H¹ NMR”) spectrum of the reaction mixture shows the desired reduction in the intensity of the olefinic proton resonances (usually about 50%), the gas flow is stopped.

The ozonolysis may be carried out as above, substituting a solution of the alkene in a solvent non-reactive towards ozone such as saturated hydrocarbons or chlorinated hydrocarbons. The ozonolysis may also be carried out as above, with or without solvent, substituting an alkenol for the alkene without affecting the reaction in any substantive manner.

The reaction mixture is then poured slowly into the cooled penetrating solvent.

### EXAMPLE 2

### Preparation of the Pharmaceutical Formulation

A preferred pharmaceutical formulation of the present invention was prepared as follows:
(1) Sparging an ozone/pure oxygen gas mixture of 120 mg/L up through an alkadiene alcohol, 3,7-dimethyl-2,6-octadien-1-ol (geraniol), at 1 Liter of gas per hour;
(2) Maintaining the temperature of the reaction around 5° C.;
(3) Removing small aliquots of reaction product hourly and measuring by H¹ NMR the formation of the peroxidic species or reaction products;
(4) Stopping the reaction when more than about 50% of the available unsaturated bonds have been reacted;
(5) Diluting the product mixture with dimethylsulfoxide (1:10) to give a solution or dispersion;
(6) Prior to use in the target biological system, a mixture of hematoporphyrin, Rose Bengal, and methyl-naphthoquinone dry powders was added to the solution or dispersion in sufficient quantity to create a concentration of 20 micromolar of each component dispersed therein when delivered to the target biological system by saline intravenous infusion. Optionally, ascorbate could be added to the formulation prior to use.

### EXAMPLE 3

### Examples of the Pharmaceutical Formulation

Two preferred formulations are as follows:

A.

| WEIGHT % | INGREDIENT |
|---|---|
| 0.54* | Tetraoxane dimer of acetal peroxide from |
| | ozonization of geraniol |
| 98.00 | DMSO |
| 0.83 | Hematoporphyrin |
| 0.24 | Methylnaphthoquinone |
| 0.39 | Rose Bengal |

*Determined by mass spectroscopy.

B.

| WEIGHT % | INGREDIENT |
|---|---|
| 0.54* | Tetraoxane dimer of acetal peroxide from |
| | ozonization of geraniol |
| 98.00 | DMSO |
| 0.83 | Hematoporphyrin |
| 0.24 | Methylnaphthoquinone |
| 0.39 | Chlorophyllin Sodium-Copper Salt |

*Determined by mass spectroscopy.

### EXAMPLE 4

### HCV Experimental Procedure

The following experiment was performed to determine whether the pharmaceutical formulation effectively treated Hepatitis C virus (“HCV”).

Five adult patients infected with HCV consented to the study. Each patient had a history of ineffective treatments using interferon and ribavirin. Ethical concerns prevented the use of a placebo-controlled, “blinded” population.

Each patient was administered the pharmaceutical formulation in twelve intravenous (“IV”) treatments. For each treatment, a non-filtered IV line with a drip chamber was attached to a 100 cc bag of sterile 0.9% saline. Then, 1 ml of Formulation A from Example 3 above was added to the saline solution and the bag mixture was shaken. A 20 or 22-gauge IV butterfly catheter was introduced and the solution was infused over 20 to 30 minutes. The treatments were administered for two consecutive days every other week over a twelve-week period. No other therapeutics or treatment procedures were administered during the twelve-week treatment course.

### EXAMPLE 5

### Viral Load Count

Polymerase chain reaction (“PCR”) allows the detection of the HCV RNA in the serum of infected patients. PCR test are extremely sensitive and have been designed specifically to detect HCV gene sequences in serum specimens. However, PCR measures only a specific section of the HCV RNA strand and does not determine if the HCV RNA strand is still intact and can actually reproduce. Nevertheless, PCR is the industry standard for determining the severity of the HCV infection.

Pre-Treatment PCR values were measured for each patient prior to beginning the treatment procedure described in Example 4. Post-treatment PCR values were measured after completion of the twelve-week treatment procedure. The viral load count for each patient is shown below in Table 5-1.

TABLE 5-1
HCV Viral Load Count

| | Pre-Treatment | Post-Treatment | Percent |
|---|---|---|---|
| Patient | (I.U./ml) | (I.U./ml) | Reduction |
| 1 | 3,700,000 | 119,000 | 97% |
| 2 | 5,620,000 | 368,600 | 93% |
| 3 | 850,000 | 592,000 | 30% |
| 4 | 7,585,200 | 4,735,800 | 38% |
| 5 | 600,000 | 288,000 | 52% |

Amongst the five patients, the average reduction in HCV viral loads was 62% after the twelve week treatment procedure. Patients 4 and 5, who are classified as Genotype 1a, the most difficult form of HCV to treat, showed viral load reductions of 38% and 52%.

In addition, all five patients demonstrated and described reductions in complaints related to fatigue, blood sugar control, weight control, circulatory problems, and joint pain, and reported overall improvements in their lifestyle and quality of life.

### EXAMPLE 6

### Liver Enzyme Function

Elevated levels of the liver enzymes alanine transaminase (“ALT”) and aspartamine transaminase (“AST”) are common in HCV-infected patients and may persist for years as acute infection enters a chronic phase. ALT and AST are released when liver cells are injured or die. The normal range of ALT in a healthy individual is 5 IU/L to 60 U/L. The normal range of AST in a healthy individual is 5 IU/L to 43 IU/L. However, it should be noted that these values vary over the course of HCV infection and may not produce an accurate forecast of disease progression.

Liver enzymes were tested before and after the twelve-week treatment procedure described in Example 4 for Patients 2-5. The results are shown in Table 6-1 below.

TABLE 6-1
Liver Enzyme Levels

| Pa- | ALT Level (I.U./L) | | AST Level (I.U./L) | |
|---|---|---|---|---|
| tient | Pre-Treatment | Post-Treatment | Pre-Treatment | Post-Treatment |
| 2 | 26 | 22 | 31 | 38 |
| 3 | 94 | 59 | 73 | 50 |
| 4 | 96 | 44 | 110 | 38 |
| 5 | 69 | 48 | 70 | 56 |

All patients showed a decrease in enzyme levels or a trend toward normalization. Patients 2-5 maintained normal levels of liver enzymes.

### EXAMPLE 7

### In Vitro Inhibition of Murine Retrovirus

The following experiment analyzed whether the pharmaceutical formulation could effectively inhibit the replication of a murine retrovirus (MoMuLV). The retrovirus MoMuLV causes thymic atrophy, neural degeneration, cachexia, cancer, and immunodeficiency.

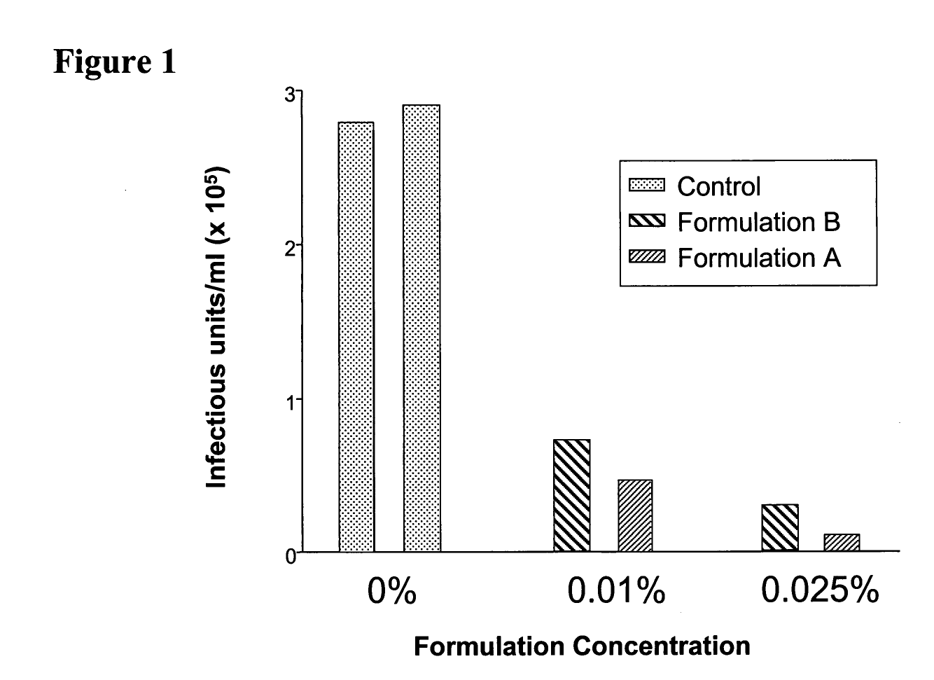

The in vitro study utilized cultured astrocyte C1 cells. The C1 cells were plated at 2.4×10⁴ cells/ml using 10% FBS DMEM medium on culture plates. After an overnight culture period, half of the plates were infected with the virus at a multiplicity of infection (“MOI”) of 4, or 4 infectious particles per cultured cell. Then, the viral solution was aspirated from the plates and replaced with one of the two pharmaceutical formulations (Formulations A and B) from Example 3, at 0.01% and 0.025%. Controls include uninfected plates and infected plates without the pharmaceutical formulation. At 24 hours after infection, 1 ml of the supernatant fluid from the plates was removed and the viral titer, or infectious units per ml, was calculated. The viral titer was calculated with standard lab procedures using 15F cells, which are from a non-transformed, non-producer, murine sarcoma-positive, leukemia-negative cell line. The 15F cells were infected with the collected supernatant from the C1 cells and cultured. The foci were counted 4-5 days later. The results of the viral titer are shown in FIG. 1.

The results indicate that the pharmaceutical formulation caused a rapid cessation of retroviral replication after only one dose. The 0.025% concentration appeared to be slightly more effective than the 0.01% concentration.

### EXAMPLE 8

### Cytotoxicity of Pharmaceutical Formulation

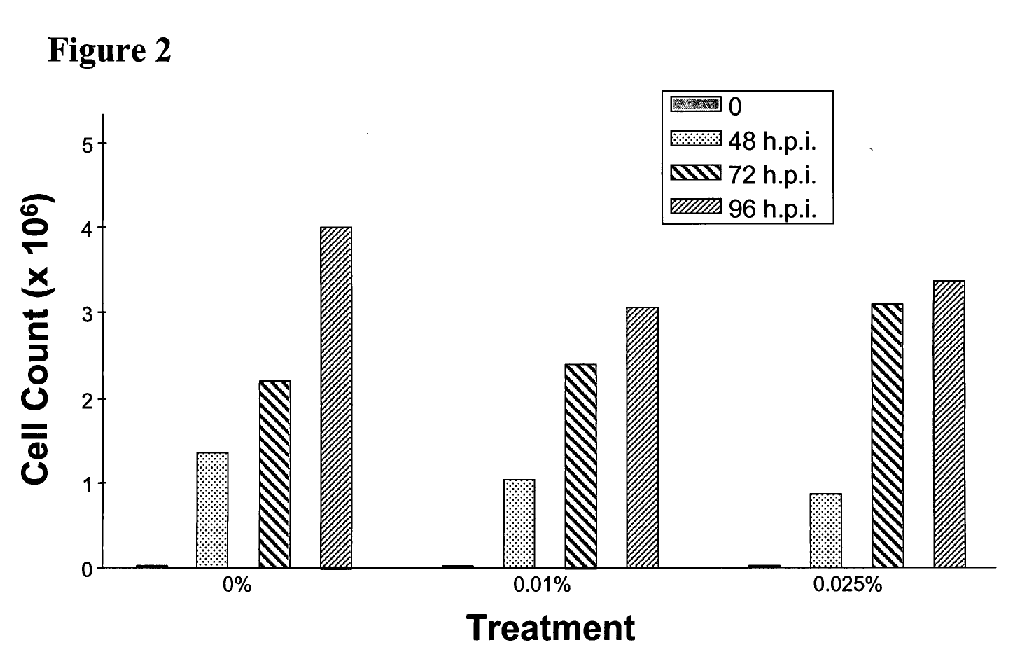

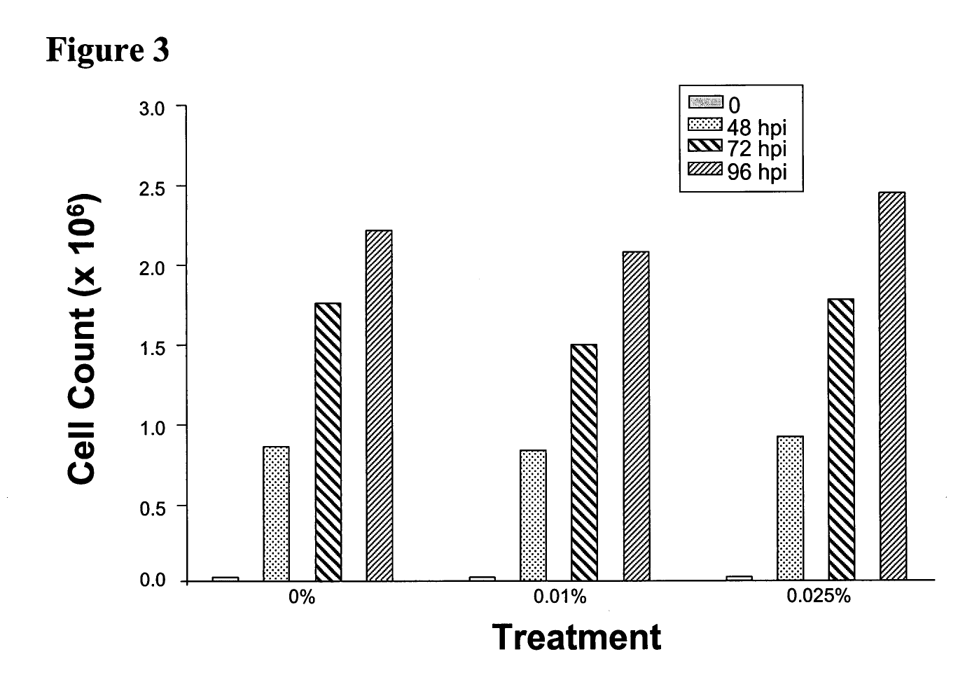

The in vitro experiment of Example 7 was also used to test the cytotoxicity of the pharmaceutical formulation on both uninfected and MoMuLV-infected C1 cells. The infection procedure was carried out as described above, with plates of uninfected cells also receiving treatments with both of the pharmaceutical formulations of Example 3 (Formulation A and B) at concentrations of 0.01% and 0.025%. At 48, 72, and 96 hours post-infection (h.p.i.), the C1 cells on the culture plates were counted and compared to controls. The results for the uninfected C1 cells are shown in FIG. 2. Cell counts for the uninfected cells were taken at the same time as the infected cells and are thus shown in hours post-infection (h.p.i.) as well. The results for the infected C1 cells are shown in FIG. 3.

The results indicate that neither of the pharmaceutical formulations were toxic for either uninfected C1 cells or C1 cells infected with the MoMuLV virus.

### EXAMPLE 9

### Suppression of HIV-1 (IIIB) Replication

The following experiment was performed to test the effectiveness of the pharmaceutical formulation at reducing the level of HIV-1 infection in cultured cells.

The prototype HIV-1 isolate IIIB has been studied for many years as a typical T-cell tropic HIV-1 isolate. IIIB isolate infects human CD4+ cells and forms syncitia, or multi-nucleated giant cells, that leads to the eventual death of the human cells.

MT-2 cells (a human CD4+ cell line) were infected with IIIB isolate at a multiplicity of infection (“MOI”) of 0.01 in the presence of Formulation A of the pharmaceutical formulation described in Example 3 above. The pharmaceutical formulation used on the treated cells had a strength of 0.02%. Untreated cells were infected with IIIB isolate in the same manner, but without the pharmaceutical formulation. Control cells were “mock infected” and cultured simultaneously.

A p24 assay was used to monitor the presence of HIV-1 p24 antigen in the culture supernatants at regular intervals, measured in days post-infection (“dpi”). A higher level of p24 antigen indicated a higher level of infection. Formation of syncitia and cell deaths were also monitored at regular intervals using microscopic examination and trypan-blue exclusion methods. Table 9-1 shows the results of the p24 assay below.

TABLE 9-1
HIV-1 (IIIB) Infection (p24 ng/ml)

| MT-2 Cells | 2 dpi | 5 dpi | 8 dpi | 11 dpi | 15 dpi |
|---|---|---|---|---|---|
| Untreated | 0.02 | 2.8 | 12.2 | 39.0 | 28.8 |
| Treated | 0.04 | 3.6 | 4.8 | 12.6 | 5.0 |
| Control | 0 | 0 | 0 | 0 | 0 |

Infected cells treated with the pharmaceutical formulation had a much lower level of infection and replication of the virus, even with only one dose. However, even with the lower level of infection, syncitia was induced at 2 to 3 dpi in both untreated and treated cells and thus the level of cell death was comparable.

The results indicate that the pharmaceutical formulation inhibits the infection and replication of HIV-1 in human CD4+ cells.

### References Cited

The following U.S. Patent documents and publications are hereby incorporated by reference.

### U.S. Patents

U.S. Pat. No. 4,451,480 to DeVillez
U.S. Pat. No. 4,591,602 to DeVillez
U.S. Pat. No. 4,983,637 to Herman
U.S. Pat. No. 5,086,076 to Herman
U.S. Pat. No. 5,126,376 to Herman
U.S. Pat. No. 5,190,977 to Herman
U.S. Pat. No. 5,190,979 to Herman
U.S. Pat. No. 5,260,342 to Herman
U.S. Pat. No. 5,270,344 to Herman
U.S. Pat. No. 5,364,879 to Herman

### Other Publications

Allen, N., Clendenon, N. R., et al. Acid hydrolase and cytochrome oxidase activities in nitrosourea induced tumors of the nervous system. Acta Neuropathol (Berl), vol. 39(1), pp. 13-23, 1977.
Manns, M. P., McHutchison, J. G., Gordon, S. C., Rustgi, V. K., Shiffinan, M., Reindollar, R., Goodman, Z. D., Koury, K., Ling, M., and Albrecht, J. K. Peginterferon alfa-2b plus ribavirin compared with interferon alfa-2b plus ribavirin for initial treatment of chronic Hepatitis C: a randomized trial. Lancet, vol. 358, pp. 958-65, 2001.
White, M. T., Arya, D. V., et al. Biochemical properties of simian virus 40-transformed 3T3 cell mitochondria. J Natl Cancer Inst, vol. 54(1), pp. 245-46, 1975.

1. A method for treating a patient infected with a virus, comprising:
administering to the patient an effective amount of a pharmaceutical formulation comprising:
peroxidic species or reaction products resulting from oxidation of an alkene by an oxygen-containing oxidizing agent, wherein the alkene comprises (α-terpineol, citronellol, nerol, linalool, phytol, geraniol, perillyl alcohol, menthol, geranylgeraniol or farnesol, and wherein the hydroxyl-containing alkene is from about 0.001% to about 30% by weight of the pharmaceutical formulation;
a penetrating solvent, wherein the penetrating solvent comprises dimethylsulfoxide, sterol, lecithin, propylene glycol, or methylsulfonylmethane, and wherein the penetrating solvent is from about 50% to about 99% by weight of the pharmaceutical formulation;
a dye containing a chelated divalent or trivalent metal, wherein the dye comprises porphyrin, rose bengal, chlorophyllin, hemin, corrins, texaphrin, methylene blue, hematoxylin, eosin, erythrosin, lactoflavin, anthracene dye, hypericin, methylcholanthrene, neutral red, or fluorescein, and wherein the dye is from about 0.1% to about 30% by weight of the pharmaceutical formulation; and
an aromatic redox compound, wherein the redox compound comprises substituted or unsubstituted benzoquinone, naphthoquinone, or anthroquinone, and wherein the aromatic redox compound is from about 0.01% to about 20% by weight of the pharmaceutical formulation.

2. The method of claim 1, wherein the alkene is in a liquid form, in a solution, or in dispersion.

3. The method of claim 1, wherein the alkene is contained in a fixed oil, an ester, a fatty acid, or an ether.

4. The method of claim 1, wherein the oxygen-containing oxidizing agent comprises singlet oxygen, oxygen in its triplet state, superoxide anion, periodate, hydroxyl radical, hydrogen peroxide, alkyl peroxide, carbamyl peroxide, benzoyl peroxide, or oxygen bound to a transition element.

5. The method of claim 1, wherein the oxygen-containing oxidizing agent comprises ozone.

6. The method of claim 1, wherein the penetrating solvent is a liquid, micelle membrane, emollient, or vapor.

7. The method of claim 1, wherein the penetrating solvent is dimethylsulfoxide (“DMSO”).

8. The method of claim 1, wherein the dye comprises porphyrin, Rose Bengal, chlorophyllin, or a mixture thereof.

9. The method of claim 1, wherein the metal comprises iron.

10. The method of claim 1, wherein the metal comprises copper, manganese, tin, magnesium, or strontium.

11. The method of claim 1, further comprising an electron donor.

12. The method of claim 11, wherein the electron donor comprises ascorbic acid or a pharmaceutical salt thereof.

13. The method of claim 1, wherein the virus is Hepatitis C or HIV-1.

14. A method for treating a patient infected with a virus, comprising:
administering to the patient an effective amount of a pharmaceutical formulation comprising:
peroxidic species or reaction products resulting from oxidation of geraniol by a mixture of ozone and oxygen;
dimethylsulfoxide (“DMSO”);
a dye containing a chelated divalent or trivalent metal, wherein the dye comprises a mixture of hematoporphyrin and Rose Bengal or a mixture of hematoporphyrin and chlorophyllin; and
methylnaphthoquinone.

15. The method of claim 14, wherein the virus is Hepatitis C or HIV-1.
